(19) United States
(12) Patent Application Publication
(10) Pub. No.: US 2021/0153734 A1
(43) Pub. Date: May 27, 2021

(54) IMPROVED MEDICAL DEVICE

(71) Applicant: THE INSIDES COMPANY LIMITED, Auckland (NZ)

(72) Inventors: Andrew Robert Stevenson, Queensland (AU); Gregory Brian O'Grady, Auckland (NZ); John Bilkey Davidson, Auckland (NZ); Robert Bruce Davidson, Auckland (NZ)

(21) Appl. No.: 16/643,749

(22) PCT Filed: Aug. 29, 2018
(86) PCT No.: PCT/IB2018/056563

§ 371 Date: Mar. 2, 2020

(30) Foreign Application Priority Data
Aug. 31, 2017 (NZ) 735124

(51) Int. Cl. A61B 1/32; A61B 1/303; A61B 1/06; A61B 1/31; A61B 1/00

(52) U.S. Cl. CPC A61B 1/32; A61B 1/303; A61B 1/00066; A61B 1/31; A61B 1/00094; A61B 1/0684

(57) ABSTRACT

A device for anorectal and/or gynaecological procedures is provided in order to improve surgical access and visual field. The device has a hollow elongate body, and an opening at the proximal end, defining an open working channel in the hollow body, and a window aperture for providing access to surrounding tissue. In addition, one or more wings are dependent from the body to aid manipulation of the device. The device further includes an illumination device and a suction passageway located within the body wherein the illumination device forms at least part of a perimeter wall of the suction passageway.

### FIELD OF THE INVENTION

The present invention relates to an improved medical device. More particularly, but not exclusively, it relates to an improved medical device for anorectal and/or gynaecological procedures.

### BACKGROUND OF THE INVENTION

Anoscopes are medical devices constructed to be used in anorectal examinations and medical procedures. A medical operator may use an anoscope to dilate the anal canal and rectum of a patient to facilitate visualisation of the cavity.

For example, anoscopes and other medical devices for anorectal procedures may be used to identify and treat conditions or diseases such as anal fissures, haemorrhoids, anorectal abscesses and fistulas, rectocoeles, rectal prolapse, rectal polyps or cancer.

Traditionally, anoscopes comprise tubular bodies and a handle. During the examination or medical procedure, the operator may use an external light source such as a headlight to illuminate the anorectal region.

However, traditional anoscopes may provide limited visualisation of the desired operating field and/or unreliable illumination. For example, an operator may wear a lighted headset which may not align ideally with the angle of the operating field. Additionally, traditional anoscopes usually lack efficiency due to ergonomics and may not offer efficient stability during medical procedures. Similar, issues may be encountered in gynaecological procedures. Excessive or rapid dilation of the anal sphincter may cause damage or discomfort.

It may be desirable for instruments used in anorectal and/or gynaecological procedures to balance a patient's safety and minimise discomfort with a medical operator's need for visualisation of the area and improved ease of use.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

For the purpose of this specification, where method steps are described in sequence, the sequence does not necessarily mean that the steps are to be chronologically ordered in that sequence, unless there is no other logical manner of interpreting the sequence.

It is an object of the present invention to provide an improved medical device for anorectal procedures which overcomes or at least partially ameliorates some of the abovementioned disadvantages or which at least provides the public with a useful choice. cl BRIEF DESCRIPTION OF THE INVENTION

According to a first aspect the invention broadly comprises a device for anorectal and/or gynaecological procedures comprising:
a hollow elongate body having a proximal end and a distal end, the body comprising an inner wall surface, and an opening at the proximal end, defining an open working channel in said hollow body, and a window aperture providing access to surrounding tissue,
one or more wings dependent from the body to aid manipulation of the device,
an illumination device located within the body, and
a suction passageway located within the body having a perimeter wall, and
wherein said illumination device forms at least partially the perimeter wall of the suction passageway.

According to another aspect said one or more wings comprises a wing cavity configured to receive light and/or suction components.

According to another aspect wherein the light component and the suction component is housed in the same wing.

According to another aspect said device comprises two wings dependent from the body.

According to another aspect the light component is housed in a first wing and the suction component is housed in a second wing.

According to another aspect said wings extend generally laterally from the proximal end of the body.

According to another aspect said wings comprise a plurality of grooves to engage with external equipment.

According to another aspect said wings comprise a connector at the distal ends of said wings to engage with external equipment.

According to another aspect said connector is a hexagonal connector.

According to another aspect said suction passageway is located along a longitudinal axis of the body.

According to another aspect said illumination device is located diametrically opposed said window aperture.

According to another aspect said suction passageway comprises one or more inlets configured to provide communication between said suction passageway and working channel.

According to another aspect said inlets are spaced along said suction passageway.

According to another aspect said one or more inlets are the distal end of the body.

According to another aspect said inner wall surface of the body forms at least partially the perimeter wall of the suction passageway.

According to another aspect said inner wall surface comprises one or more wall protrusions along a longitudinal axis of said body configured to form at least partially the perimeter wall of the suction passageway.

According to another aspect said device comprises one or more inlets between said wall protrusions to provide communication between said suction passageway and working channel.

According to another aspect said suction passageway is connected to an external suction or fluid source at the proximal end of the body.

According to another aspect said suction passageway extends along a said wing.

According to another aspect said suction passageway is connected to an external suction source or fluid source at a distal end of said wing.

According to another aspect said illumination device comprises a light pipe.

According to another aspect said illumination device comprises a light-emitting diode.

According to another aspect said light pipe tapers from a proximal end of the light pipe to the distal end of the light pipe.

According to another aspect said inner wall surface comprises measurement markings configured to indicate distance.

According to another aspect said measurement markings are protrusions on the inner wall surface configured to cast a shadow on the inner wall surface when the illumination device is on.

According to another aspect said device further comprising a tapered head at the distal end of the body to aid insertion of the device.

According to another aspect said device further comprising a flared end at the proximal end of the body for stability of the device in use.

According to another aspect said window aperture is an elongated opening.

According to another aspect said window aperture extends from the proximal end of the body to the distal end of the body.

According to another aspect said body has a semi-circular cross section.

According to another aspect said inner wall surface comprises a rough surface to disperse light from the illumination device along the body.

According to another aspect said illumination device can be turned on or off.

According to another aspect said device comprises a switch or tab to turn the illumination device on or off.

Other aspects of the invention may become apparent from the following description which is given by way of example only and with reference to the accompanying drawings.

As used herein the term “and/or” means “and” or “or”, or both.

As used herein “(s)” following a noun means the plural and/or singular forms of the noun.

The term “comprising” as used in this specification and claims means “consisting at least in part of”. When interpreting statements in this specification and claims which include that term, the features, prefaced by that term in each statement, all need to be present but other features can also be present. Related terms such as “comprise” and “comprised” are to be interpreted in the same manner.

### BRIEF DESCRIPTION OF THE DRAWINGS

The invention will now be described by way of example only and with reference to the drawings in which:

FIG. 1 shows a perspective view of the anorectal device.

FIG. 2 shows a cross section the body of the anorectal device on a transverse plane.

FIG. 3 shows an exploded view of a cross section of the anorectal device.

FIG. 4 shows another cross section of the anorectal device.

FIG. 5 shows an exploded view of the anorectal device.

FIG. 6 shows a perspective view of the anorectal device with the light pipe removed.

FIG. 7 shows another perspective view of the anorectal device.

FIG. 8 shows a cross section of the anorectal device with a light circuit and suction connection.

FIG. 9 shows a cross section on the frontal plane of the anorectal device with a suction connector.

FIG. 10 shows partial perspective close-up view of the distal end of a wing.

### DETAILED DESCRIPTION OF PREFERRED EMBODIMENTS

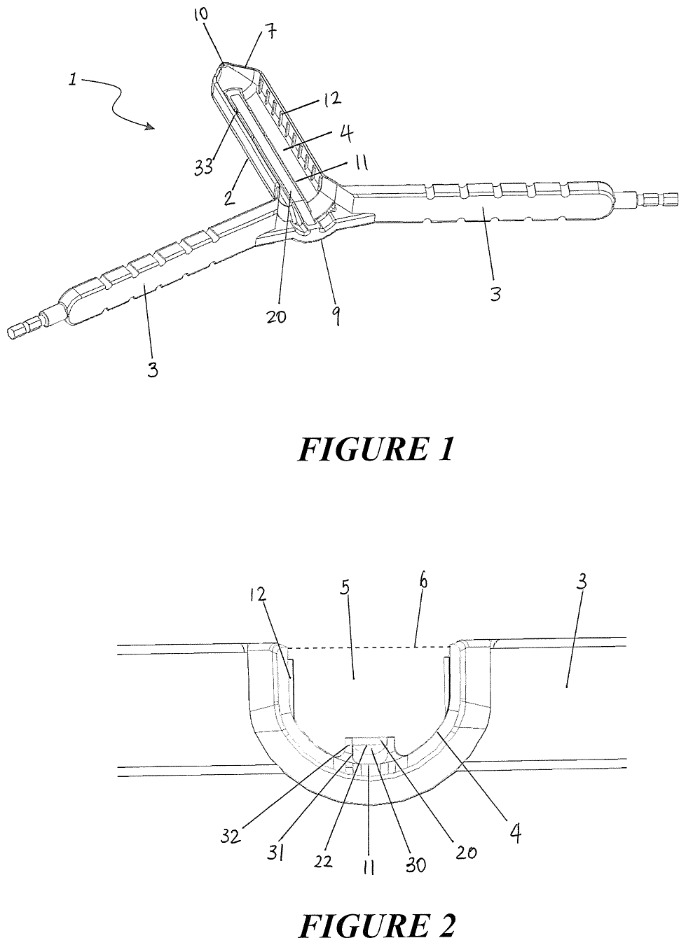

With reference to FIGS. 1-10 there is shown an anorectal device 1 configured to provide access to and aid visualisation of the anorectal region of a patient to examine and/or treat a range of anorectal conditions and diseases.

It is also anticipated that the device 1 can be used in other medical procedures requiring assess to elongate cavities such as vaginal examination and procedures. It may be preferable to adopt different dimensions to suit a gynaecological applicator , but the general shape may remain.

With reference to FIG. 1, the anorectal device 1 comprises a hollow elongate body 2, and one or more wings 3 dependent from the body. The anorectal device 1 preferably comprises an integrated illumination device 20 and suction passageway 30 to be discussed in more detail below.

The elongate body 2 is adapted to be inserted into the anal canal and rectum of a patient or a similar elongate cavity. The body 2 of the anorectal device 1 is preferably shaped and sized to provide unimpeded procedural access and viewing of the anorectal region which generally has confined viewing. The anorectal device 1 hold up the internal cavity walls as the device retracts the anorectal passage of a patient or other potentially obstructing objects such as an internal rectal prolapse, a haemorrhoid plexus, or a mucosal fold of a patient during the examination or medical procedure.

Optionally, the body 2 of the anorectal device 1 comprises a smooth outer surface for provide smooth entry into a cavity. Alternatively the body 2 of the anorectal device 1 comprises textured outer surface or grooves to provide slip-resistance or friction between the device and the anorectal cavity.

Preferably, the elongate body 2 is long enough to provide access and visualisation of the low-mid rectum region. In the most preferred configurations, the elongated body is between 100 mm and 200 mm long, although it may be longer. For gynaecological applications, the total length may be shorter and/or the diameter larger.

The body 2 in its preferred configurations is approximately cylindrical. Preferably the body 2 of the device 1 has an approximately semi-circular cross section in the transverse plane as shown in FIG. 2. In other configurations, the body 2 has a partially circular, elliptical or partially elliptical cross section.

The elongate body 2 has a proximal end 9 and a distal end 10. The proximal end 9 is the end directed towards a medical operator when the anorectal device 1 in use and held at the wings 3. The distal end 10 is the end opposite the proximal end 9 and is further from the medical operator than the proximal end when the anorectal device 1 is in use.

With reference to FIGS. 1 and 2, the body 2 of the anorectal device 1 preferably includes an inner wall surface 4. The inner wall surface 4 preferably forms an open working channel 5. The open working channel 5 is sized and configured to be inserted into the anorectal region while providing free working space which may be used by a medical operator during medical procedures. The open working channel 5 allows operators to perform medical procedures without obstruction by other parts of the device 1.

In the preferred configuration, the diameter of the open working channel 5 is between approximately 29 mm and 33 mm, for anorectal applications, although anywhere between approximately 20 mm and 40 mm is envisaged.

For gynaecological applications the diameter may be larger and up to approximately 50 mm, or more.

Optionally, the open working channel 5 facilitates insertion of medical instruments necessary for some medical procedures. For example, a needle holder and suture, an instrument for haemorrhoidal treatment (e.g. band ligation applicator), a fistula probe, scalpel, diathermy, scissors, needle for injections, grasping forceps, or biopsy forceps may be inserted into the open working channel 5 of the device 1 during medical procedures.

The body 2 preferably includes a window aperture 6 as best illustrated as a dashed line in FIG. 2. The window aperture 6, is preferably located opposite the base 11 of the inner wall surface 4.

The window aperture 6 preferably provides access to tissue surrounding the anorectal device 1 from the open working channel 5 when the device has been inserted in a cavity.

In the preferred configurations, the window aperture 6 is an elongated opening. Preferably, the window aperture 6 is formed along a longitudinal axis of the body 2. Preferably, the window aperture 6 is sized to allow a medical operator space to suture a wound or ‘purse-string’ suture on the surrounding tissue.

In some configurations, the window aperture 6 extends from the proximal end 9 of the body 2 to the distal end 10 of the body. In other configurations, the window aperture 6 extends partially along the proximal end 9 and/or the distal end 10 of the body 2.

Preferably, the window aperture 6 provides a wide field to visualise the surrounding tissue. In the preferred configuration, the window aperture 6 comprises a window width spanning the whole diameter of the body 2 of the anorectal device 1. In alternative configurations, the window aperture 6 comprises a window width less than the diameter of the body 2.

At the distal end 10 of the device 1, the body 2 preferably comprises a tapered or rounded head 7 configured to aid insertion of the device into the anorectal (or vaginal) region. As the anal canal and rectum of a patient is generally biased closed, introduction of a device 1 with a gradually increasing diameter from the distal end 9 may provide atraumatic entry by minimising tissue injury or patient discomfort during insertion of the device. A tapered head 7 may also decrease patient discomfort when the anorectal device 1 is being inserted. Gradual dilation of the anorectal cavity may be desired because the device 1 is inserted into a region where there are many nerve endings and the anal sphincter may be vulnerable to damage by excessive rapid stretching.

In the preferred configurations, the proximal end 9 of the anorectal device 1 comprises a flared end 8 where the width of the flared end region is greater than the width of the open working channel 5 as best shown in FIGS. 1 and 8. An advantage of an anorectal device 1 with a flared end 8 is that it may improve stability of the device 1 as it sits against the anal verge when the device is in use. Additionally, the flared end 8 of the anorectal device 1 may prevent the device from penetrating into the anal cavity further than desired as the flared end 8 engages with the anal verge.

In the preferred configurations, the anorectal device 1 comprises an illumination device 20. Preferably the illumination device 20 is inbuilt and integral with the device 1 to eliminate the need to provide an external lighting device. An inbuilt illumination device 20 may reduce a medical operator's discomfort and strain during examinations and medical procedures as the operator will not need to wear a headlight or hold a hand-held light device. An integrated illumination device 20 may also provide better illumination of the working field in comparison to ceiling-mounted theatre lighting systems, as that system may limit angles at which the working field may be viewed at. An integrated illumination device 20 may also eliminate shadows cast in headlight, hand-held light or ceiling-light systems due to the potential obstruction of the light source by an operator or medical instruments.

An integrated light source also allows the light source to be low profile, thus maximising the space for the surgeon.

Preferably the illumination device 20 is located within the body 2 of the device. Preferably, the illumination device 20 is located along the longitudinal axis of the body 2. Alternatively, the light 20 is located along the transverse axis of the body 2, or both the transverse and longitudinal axis of the body.

In the preferred configurations, the illumination device 20 is a light pipe. The light pipe 20 preferably emits and disperses light along the length of the working channel 5. Light dispersed along the whole length of the working channel 5 may be advantageous as this feature may minimise the need to reposition the anorectal device 1 along the cavity once inserted, to best light up tissue surrounding the device requiring examination or surgery. In other configurations, the light pipe 20 extends partially along the length of the body 2.

In the preferred configurations the light pipe 20 is located towards the base 11 of the inner wall surface 4 of the anorectal device 1. Preferably the light pipe is located diametrically opposed to the window aperture 6.

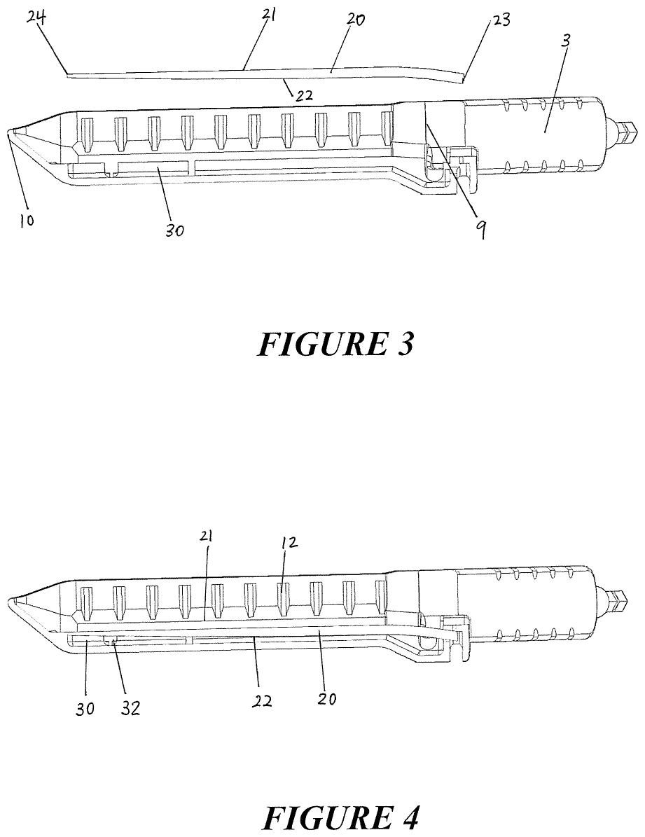

An upper surface 21 of the light pipe 20, best shown in FIGS. 3 and 4 faces the open working channel 5. The upper surface 21 of the light pipe 20 preferably comprises a flat profile to help maximise the area of the open working channel 5 for the medical operator to utilise. A light pipe 20 comprising a flat upper surface 21 may also be advantageous as medical instruments used in the procedure may not get caught on the light pipe 20.

A lower surface 22 of the light pipe 20, faces the base 11 of the inner wall surface 4.

In the most preferred configurations, the light pipe 20 tapers (in thickness) from a proximal end 23 of the light pipe, to the distal end 24 of the light pipe, as best illustrated in FIG. 4. A tapered light pipe 20 emits light more evenly along the working channel 5. A tapered light pipe 20 emits light in a more uniform and controlled manner from the upper surface 21 of the light pipe. A tapered light pipe 20 scatters light upwards into the open working channel 5, and may minimise or eliminate glare where light may be reflected towards an operator at the proximal end 9 of the device 1.

Preferably, the inner wall surface 4 comprises a rough surface so that light bounces along the open working channel 5 to disperse light from the illumination device 20 along the body 2 of the device 1.

Preferably the light source is a light-emitting diode (LED) light pipe which may help provide uniform illumination, reduce glare, while remaining low profile, and may be easy installed into the anorectal device 1 during manufacture.

Optionally, the light source 20 comprises a disposable light-pipe and battery which can be replaced as required.

It is anticipated that other illumination device 20 can be used such as optical fibres.

Optionally, different light frequencies may be used in the light pipe 20. For example tumours may be better detected by using narrow-band imaging.

Optionally, the device 1 includes a multi-frequency light source to allow the operator to choose between different light frequencies during the procedure. For example, narrow-band imaging light frequencies for assessing luminal pathologies, specific light spectrums in conjunction with fluorescence for image-guided surgery or light frequencies for hyperspectral imaging may be available.

Preferably, the lighting component 20 can be turned on or off. In some configurations, the anorectal device 1 comprises a control switch or tab to turn the lighting component 20 on or off (not shown), and/or to change the emitted light frequency (if available).

In the preferred configurations, the anorectal device 1 comprises a suction passageway 30. The suction passageway 30 is adapted to provide a path for fluids to travel away from the working channel 5 to be safely disposed. Suction may help reduce or remove unwanted fluid such as blood, mucus, pus, surgical wash or diathermy smoke.

Preferably, the suction passageway 30 is removably connected to an external suction source, for example a pump (not shown) to remove fluid.

Preferably, the suction passageway 30 is connected by standard suction tubing to a collection canister.

Optionally, the suction passageway 30 is connected to the external suction source at the proximal end 9 of the body 2 of the device 1, for example a wall-mounted vacuum attachment traditionally provided in surgical theatres.

Optionally, the suction passageway 30 may be used for irrigation.

Extraction of fluids/smoke may be desired to improve visualisation of in the working channel 5 during examinations and other medical procedures. Extraction of smoke from the working channel 5 during processes such as trans-anal excisions may be particularly advantageous to minimise smoke inhalation and other medical hazards and/or to improve view of the working field for medical operators. For example, extraction of smoke from the working channel 5 may minimise the potential risk of aerosolising HPV virus, posing as an infection risk to medical staff during surgery on condyloma acuminate.

Preferably the suction passageway 30 is located with the body 2 of the device. Preferably the suction passageway 30 is located along the longitudinal axis of the body 2.

With reference to FIG. 2, a perimeter wall 31 defines the perimeter of the suction passageway 30.

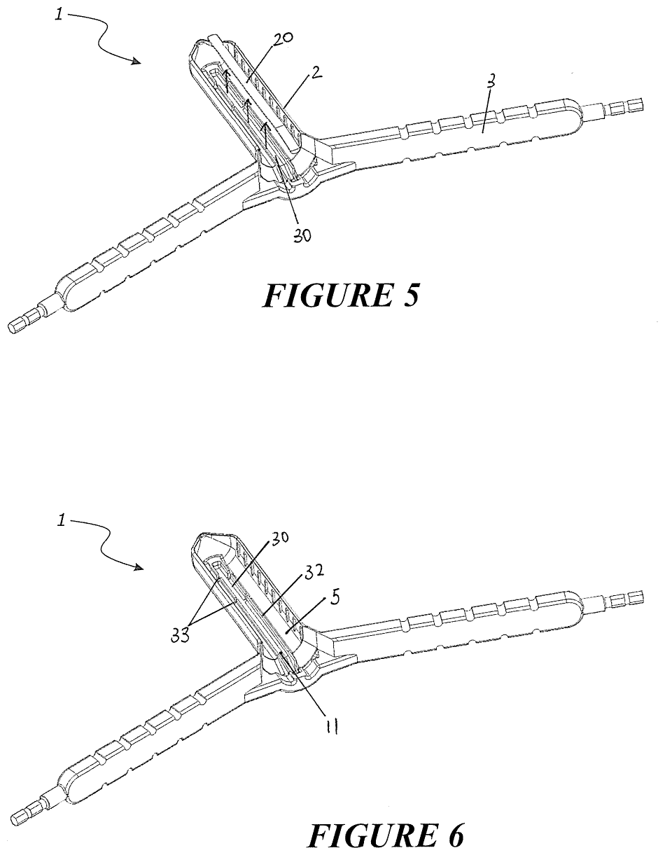

Preferably, the suction passageway 30 is located beneath the light pipe 20 as illustrated in FIGS. 5 and 6.

In the preferred configurations the suction passageway 30 is located towards or at the base 11 of the inner wall surface 4 of the anorectal device 1. Preferably the inner wall surface 4 of the body 2 forms at least partially the perimeter wall 31 of the suction passageway 30.

In the most preferred configurations, the bottom surface 22 of the light pipe 20 forms a remaining part of the perimeter wall 31 of the suction passageway 30 as best shown in FIGS. 2 and 4. It may be advantageous to integrate the light pipe 20 with the suction passageway 30 to provide a flatter and more compact profile within the body 2 of the device 1. A flatter profile within the body 2 of a device 1 for anorectal procedures may provide for a more compact device.

An integrated light pipe 20 and suction passageway 30 leaves space in the body 2 of the device 1 for a larger open working channel 5. A larger open working channel 5 may be desired by medical operators as it may provide them with more space to manipulate medical equipment in the operating field during some medical procedures, including suturing. In addition, a reduction in the number of parts may be achieved.

In the preferred configurations, the inner wall surface 4 of the anorectal device 1 forms part of the perimeter wall 31 of the passage way 30.

With reference to FIGS. 1, 2 and 6, the anorectal device 1 optionally comprises one or more side wall protrusions 32 from the inner wall surface 4 of the device, to form part of the side perimeter wall 31 of the suction passageway 30. Preferably the protrusions 32 are located along the longitudinal axis of the body 2 of the device 1.

In some configurations, the anorectal device 1 comprises a plurality of wall protrusions 32 along the length of the body 2.

Preferably, the suction passageway 30 comprises one or more inlets 33 where fluid enters the suction passageway from the working channel 5 of the anorectal device 1. Preferably, the inlets 33 provide communication between the suction passageway 30 and working channel 5.

Preferably, the one or more inlets 33 are located towards the distal end 30 of the body 2 of the anorectal device 1. In other configurations the one or more inlets 33 are located along the whole length of the body 2 of the anorectal device 1.

In some configurations, a plurality of inlets 33 are formed from openings between a plurality of wall protrusions 32 is best shown in FIG. 6.

In some configurations, the passageway 30 is used as a path to deliver fluid for irrigation and/or delivery of other medicaments. When the anorectal device 1 is used to deliver fluids, openings in the passageway 30 are outlets 33 for fluid to be delivered from the passageway to the open working channel 5.

An anorectal device 1 comprising an integrated suction passageway 30 may be advantageous as a separate external suction device may not be necessary. An anorectal device 1 comprising an inbuilt suction passageway 30 may free an operator's or assistant's hands during medical procedures to perform other tasks. This may be helpful in anorectal procedures where medical operators are working in a confined operating space. Insertion of an external suction device into the open working channel 5 may decrease the working space available and visualisation, while an inbuilt suction passageway 30 will not have the same disadvantage. In addition, where the light pipe 20 is preferably removable from the body 2, the device may be easier to clean and/or sterilise.

In the most preferred configurations, the anorectal device 1 comprises two wings 3 dependent from the body 2 of the device. A device 1 comprising two wings 3 may facilitate smoother manipulation of the device in sensitive regions.

Preferably, the wings 3 extend laterally from the proximal end 9 of the body 2 of the anorectal device 1.

In some configurations, the wings 3 comprise a substantially rectangular cross-section. It is anticipated that the wings 3 can be of a different shape, for example the wings 3 comprise a circular cross-section in other configurations.

The wings 3 are adapted to be held by an operator. The wings 3 may be used by the operator to manipulate the device 1 into different desired positions. Preferably the wings 3 act as a handle to assist in rotational and translational positioning of the device 1. Rotation and translation of the device may be necessary to position the window aperture 6 to the examination or treatment site.

Preferably the wings 3 are approximately 150 mm to 200 mm long. The wings 3 are preferably are long enough to allow the operator or an assistant to ergonomically hold the device 1 without obstructing the operating field.

Optionally the wings 3 have a rough surface, or notches to facilitate gripping by the medical operator.

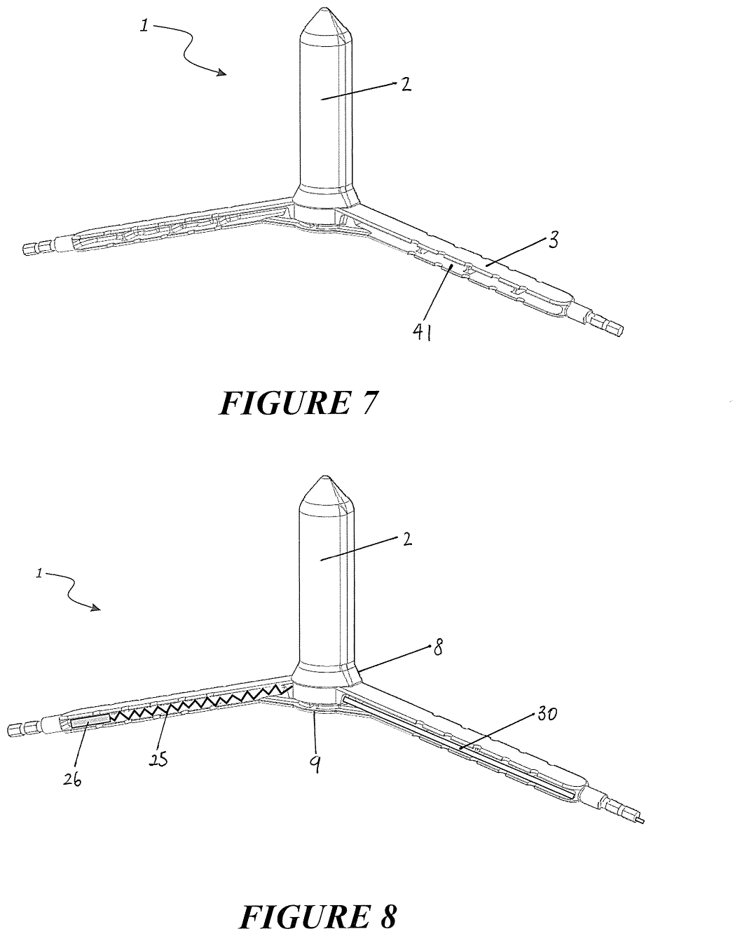

With reference to FIG. 7, in the preferred configurations, the wings 3 are hollow, and each comprise a cavity 41. Wings 3 comprising a cavity 41 are adapted to house components of the device.

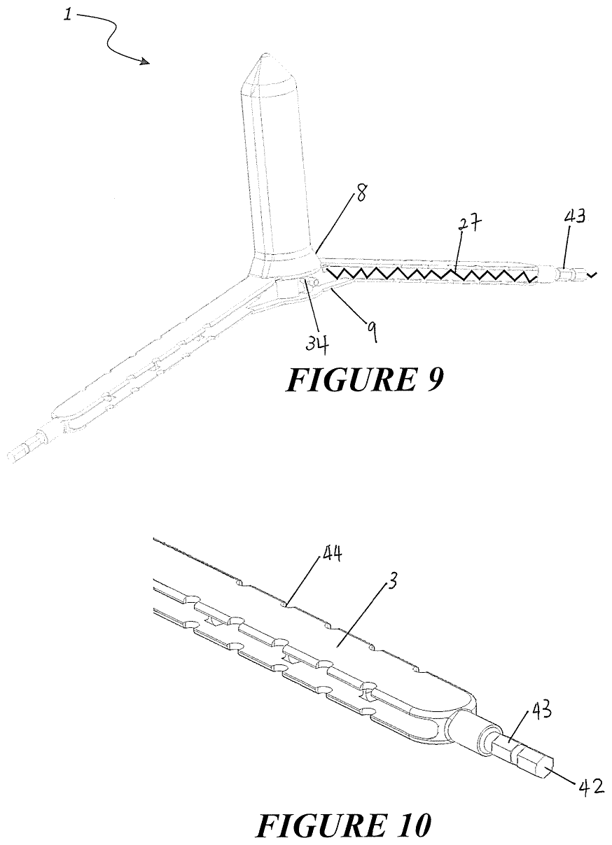

In the preferred configurations, the wings 3 comprises a cavity 41 adapted to receive a light component 25. In some configurations, the wings 3 house a power source 26 such as a battery and/or LED driver, as shown in FIG. 8. In other configurations, the light pipe 20 is connected to an external power source. Optionally, the wings 3 house the light circuit 27 of the light pipe 20 adapted to be connected to an external power source as illustrated in FIG. 9. In this configuration, the light circuit 27 preferably exits through a distal end 42 of the wing 3.

In the preferred configurations, a wing cavity 41 is adapted to receive a suction component. The suction component is preferably an extension of the suction passageway 30. Optionally, the extension of the suction passageway 30 comprises suction tubing (not shown) which runs along the length of a wing 3. In this configuration, the suction passageway 30 preferably exits through the distal end 42 of the wing 3. Optionally, the suction tubing can be secured in the wing cavity 41 using adhesive, clips, locks or other fasteners.

Optionally, in another configuration, the anorectal device 1 comprises a suction connector piece 34 adapted to removably connect the suction passageway 30 with an external suction source. In some configurations, the connector piece 34 is located at the proximal end 9 of the body 2 as illustrated in FIG. 9.

In the most preferred configurations, the wing cavity 41 is adapted to receive and house both a light component 25 and a suction component 30.

In one configuration, the light component 25 and the suction component 30 are housed in the same wing 3. In another configuration, the light component 25 is housed in a first wing and a suction component 30 is housed in a second wing.

Housing of light and suction components in the wing cavity 41 may be desired as it may decrease or eliminate the need to hold these components separately from the anorectal device 1. Housing of light and suction components may also be desired as it may decrease or eliminate the likelihood of tangling these components during examination or medical procedures.

Optionally, the wing cavity 41 comprises internal pillars between the top and bottom walls of the wings 3 for strengthening of the wings.

In the most preferred configurations, the anorectal device 1 is configured to be connected to external medical equipment to stabilise the device especially during more complex anorectal procedures, this may be especially advantageous when an assistant is not available during a medical procedure. An advantage of connecting the device 1 to external stabilisation equipment is that once the device is in place, the operator may let go of the device, and may be able to attend to other tasks for the medical procedure.

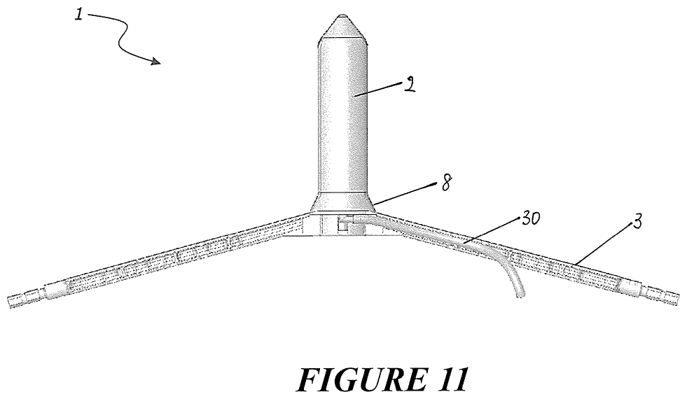

In some configurations, the anorectal device 1 comprises a connector 43 at the distal end 42 of the wing 3 adapted to be connected to external medical equipment as best shown in FIG. 10. For example, the connector 43 is a hexagonal connector adapted to be connected to surgical holder arms such as a Mediflex™ table arm.

In some configurations, the anorectal device 1 comprises a plurality of grooves 44 along the wings 3 of the device. The grooves 44 along the wings are adapted to allow interfacing with external retracting system frames, such as a Lone-Star™ retractor system by using rubber slings.

Optionally, the anorectal device 1 comprises measurement markings 12 configured to indicate distance along the length of the body 2 of the device.

Preferably, the measurement markings 12 are protrusions on the inner wall surface 4 of the device. The measurement markings are protrusions 12 protrude into the open working channel 5. Preferably, the measurement marking protrusions 12 cast a shadow on the inner wall surface 4 of the device when the illumination device 20 is on.

Optionally, the measurement marking protrusions 12 comprises a different material or colour from the body 2 of the anorectal device 1 for better visualisation of the measurement markings.

Advantages of the measurement markings 12 may include aiding measurement of distances from a pathology to the anal verge or providing a guide for placing accurate circumferential ‘purse-string’ sutures in medical procedures such as a trans-anal total mesorectal excision (TaTME) surgery.

Optionally, the anorectal device 1 comprises a camera (not shown) directed at the window aperture 6 of the device. The camera may transmit images or video so that the examination or medical procedure can be viewed in real time or retained to be viewed after the procedure.

The anorectal device 1 is preferably formed from a suitable polymer material. In some embodiments, the device 1 may be of a transparent plastic to allow visualisation of a greater area of the surrounding tissue.

Alternatively, the anorectal device 1 is formed from a metal, for example stainless steel. Preferably, the stainless steel has a dull finish to minimise reflection of light off the device 1 during a medical procedure.

However other suitable materials with sufficient stiffness and durability may be used.

In the most preferred configurations, the body 2 of the anorectal device 1 is predominantly integrally formed as one unit. In some configurations, the anorectal device 1 comprises a disposable light pipe and/or battery.

A one unit device (or a device with a reduced number of parts) with features such as integrated illumination and suction may be advantageous as it may be more easily sterilised. Sterilisation of medical equipment is important as the device may be reused for multiple procedures and there is generally a need to minimise or prevent the risk of transmitting germs or corrosion of the device or other medical equipment it is in contact with. A one unit device may be advantageous as it may be easily manipulated during the procedure without having to reposition external components such as illumination or suction devices. Furthermore, a device with integrated illumination and suction may be desired, as it is ergonomically designed to provide more operating space, and free up operator's and assistant's hands for other tasks.

To those skilled in the art to which the invention relates, many changes in construction and widely differing embodiments and applications of the invention will suggest themselves without departing from the scope of the invention as defined in the appended claims.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

1. A device for anorectal and/or gynaecological procedures comprising:
a hollow elongate body having a proximal end and a distal end, the body comprising an inner wall surface, and an opening at the proximal end, defining an open working channel in said hollow body, and a window aperture for providing access to surrounding tissue,
one or more wings dependent from the body to aid manipulation of the device,
an illumination device located within the body, and
a suction passageway located within the body having a perimeter wall, and
wherein said illumination device forms at least a part of the perimeter wall of the suction passageway.

2. A device for anorectal and/or gynaecological procedures as claimed in the previous claim, wherein one or more wings comprises a wing cavity configured to receive light and/or suction components.

3. (canceled)

4. A device for anorectal and/or gynaecological procedures as claimed in claim 2, wherein said device comprises two wings dependent from the body.

5. A device for anorectal and/or gynaecological procedures as claimed in the previous claim, wherein the light component is housed in a first wing and the suction component is housed in a second wing.

6. A device for anorectal and/or gynaecological procedures as claimed in claim 1, wherein said wings extend generally laterally from the proximal end of the body.

7.-9. (canceled)

10. A device for anorectal and/or gynaecological procedures as claimed in claim 1, wherein said suction passageway is located along a longitudinal axis of the body.

11. A device for anorectal and/or gynaecological procedures as claimed in claim 1 wherein said illumination device is located diametrically opposed said window aperture.

12. A device for anorectal and/or gynaecological procedures as claimed in claim 1, wherein said suction passageway comprises one or more inlets configured to provide communication between said suction passageway and working channel.

13. A device for anorectal and/or gynaecological procedures as claimed in claim 12, wherein said one or more inlets are spaced along said suction passageway.

14. A device for anorectal and/or gynaecological procedures as claimed in claim 13, wherein said one or more inlets are at a distal end of the body.

15. A device for anorectal and/or gynaecological procedures as claimed in claim 1, wherein said inner wall surface of the body forms at least partially the perimeter wall of the suction passageway.

16. A device for anorectal and/or gynaecological procedures as claimed in claim 1, wherein said inner wall surface comprises one or more wall protrusions along a longitudinal axis of said body configured to form at least partially the perimeter wall of the suction passageway.

17. A device for anorectal and/or gynaecological procedures as claimed in claim 16, wherein said device comprises one or more inlets between said wall protrusions to provide communication between said suction passageway and working channel.

18. (canceled)

19. A device for anorectal and/or gynaecological procedures as claimed in claim 1, wherein said suction passageway extends along a said wing.

20. (canceled)

21. A device for anorectal and/or gynaecological procedures as claimed in claim 1, wherein said illumination device comprises a light pipe.

22. (canceled)

23. A device for anorectal and/or gynaecological procedures as claimed in claim 21, wherein said light pipe has a thickness that tapers from a proximal end of the light pipe to the distal end of the light pipe.

24. A device for anorectal and/or gynaecological procedures as claimed in claim 1, wherein said inner wall surface comprises measurement markings configured to indicate distance.

25. A device for anorectal and/or gynaecological procedures as claimed in claim 24, wherein said measurement markings are protrusions on the inner wall surface configured to cast a shadow on the inner wall surface when the illumination device is on.

26. A device for anorectal and/or gynaecological procedures as claimed in claim 1, said device further comprising a tapered head at the distal end of the body to aid insertion of the device.

27. A device for anorectal and/or gynaecological procedures as claimed in claim 1, said device further comprising a flared end at the proximal end of the body for stability of the device in use.

28. A device for anorectal and/or gynaecological procedures as claimed in claim 1, wherein said window aperture is an elongated opening.

29. A device for anorectal and/or gynaecological procedures as claimed in claim 28, wherein said window aperture extends from the proximal end of the body to the distal end of the body.

30. A device for anorectal and/or gynaecological procedures as claimed in claim 1, wherein said body has a semi-circular cross section.

31. A device for anorectal and/or gynaecological procedures as claimed in claim 1, wherein said inner wall surface comprises a rough surface to disperse light from the illumination device along the body.

32.-33. (canceled)
